(19) United States
(12) Patent Application Publication
(10) Pub. No.: US 2017/0189315 A1
(43) Pub. Date: Jul. 6, 2017

(54) SYSTEMS AND METHODS FOR IMPROVING THE APPEARANCE OF SKIN

(71) Applicant: L'OREAL, Paris (FR)

(72) Inventors: Anne-Laure Suzanne BERNARD, New York, NY (US); Yang DENG, Edison, NJ (US); Alexandra Jane Elisa FARRAN, Dayton, NJ (US)

(21) Appl. No.: 15/094,259

(22) Filed: Apr. 8, 2016

Related U.S. Application Data
(60) Provisional application No. 62/274,078, filed on Dec. 31, 2015.

(51) Int. Cl. A61K 8/891; A61Q 1/02; A61K 8/92; A61Q 19/08; A61K 8/81

(52) U.S. Cl. CPC A61K 8/891; A61Q 19/08; A61K 8/8147; A61K 8/8194; A61K 8/92; A61Q 1/02; A61K 2800/10

(57) ABSTRACT

The disclosure relates to systems and methods for improving the appearance of the skin. Systems comprise a skin-tightening composition comprising at least one thermoplastic elastomer, at least one adhesive polymer, and at least one filler, and a cosmetic composition comprising at least one colorant and optionally at least one volatile solvent. Methods comprise applying the compositions of the systems on to the skin.

This application claims priority to U.S. Provisional Patent Application No. 62/274,078, filed Dec. 31, 2015.

### TECHNICAL FIELD

The disclosure relates to systems and methods for improving the appearance of the skin.

### BACKGROUND

Skin is primarily comprised of two layers. The outer layer, or epidermis, has a depth of approximately 100 μm. The inner layer, or dermis, has a depth of approximately 3000 μm from the outer surface of the skin and is comprised of a network of fibrous protein known as collagen, which provides skin firmness, and elastin, which supplies skin elasticity and rebound. As a person ages, their skin produces less collagen and elastin each year. As a result, the skin becomes thinner and more fragile with age, and wrinkle formation as a result of aging is inevitable. It is common for consumers to wish to improve the appearance of such age-related skin imperfections such as wrinkles, crow's feet, eye bags, and the like.

While some cosmetic formulations may include an ingredient to reduce the appearance of such wrinkles over time, such as an anti-wrinkle cream, such formulations may take a long time for results to be noticeable, and may also be ineffective to reduce the appearance of more pronounced skin imperfections.

As an alternate to topical cosmetic formulations, more invasive techniques such as surgery, fillers, or laser resurfacing of the skin may provide longer-lasting effects and can treat prominent imperfections. However, many consumers either cannot afford, or do not wish, to undergo such drastic cosmetic treatments.

Furthermore, as a person ages, other skin imperfections may appear or become more noticeable. For example, age spots, which are brown or gray sun-induced skin lesions, may appear on sun-exposed skin as a person gets older. Additionally, many consumers wish to improve the appearance of, or hide, other skin imperfections such as acne, scars, enlarged pores, and so on, which may not be related to aging.

While topical cosmetic formulations such as make-up, foundation, or concealer may improve the appearance of some skin imperfections, such formulations have a tendency to settle into or “mark” wrinkles, making them appear more pronounced.

As such, there is a consumer desire for topical cosmetic formulations that are effective at reducing the appearance of skin imperfections while simultaneously reducing the appearance of wrinkles.

### SUMMARY

The disclosure relates to systems and methods for improving the appearance of the skin.

In various embodiments, the disclosure relates to systems comprising: (1) a skin-tightening composition comprising (a) at least one amorphous hydrocarbon block copolymer of styrene and monomers of hydrocarbon containing 2 to 5 carbon atoms and comprising one or two ethylenic unsaturations, and having a first T_{g} below about 0° C., and a second T_{g} greater than about 25° C., (b) at least one adhesive film-forming polymer chosen from polymer particles of C₁-C₄ alkyl(methacrylate)polymer, stablilized in a non-aqueous dispersion, and (c) at least one filler; and (2) a cosmetic composition for making up the skin comprising at least one colorant and optionally at least one volatile solvent.

In further embodiments, the disclosure relates to methods for improving the appearance of the skin, said methods comprising applying to the skin a system comprising: (1) a skin-tightening composition comprising (a) at least one amorphous hydrocarbon block copolymer of styrene and monomers of hydrocarbon containing 2 to 5 carbon atoms and comprising one or two ethylenic unsaturations, and having a first T_{g} below about 0° C., and a second T_{g} greater than about 25° C., (b) at least one adhesive film-forming polymer chosen from polymer particles of C₁-C₄ alkyl(methacrylate)polymer, stablilized in a non-aqueous dispersion, and (c) at least one filler; and (2) a cosmetic composition for making up the skin comprising at least one colorant and optionally at least one volatile solvent.

### DETAILED DESCRIPTION

In various embodiments, the disclosure relates to systems and methods for improving the appearance of the skin. According to various embodiments, the systems according to the disclosure comprise a skin-tightening composition capable of forming a film on the skin, and a cosmetic composition for making up the skin, and methods comprising applying the skin-tightening composition onto the skin before or after applying the cosmetic composition onto the skin.

The systems and methods may be effective at reducing the appearance of skin imperfections. In various embodiments, the systems and methods may improve the appearance of wrinkles in the skin by forming a film on the skin that has a Young Modulus greater than that of skin, and thus has the capability of tightening the skin, while reducing the appearance of skin imperfections such as pimples, scars, age-spots, uneven skin tone, and the like, for example by blurring or hiding such skin imperfections.

As used herein, the term “long-lasting” means that the film lasts for at least about 6 hours, such as at least about 12 hours, at least about 24 hours, at least about 48 hours, or at least about 72 hours, after the film is formed on the skin.

As used herein, the term “lasting” it is meant to convey that the film is substantially intact in place on the skin.

As used herein, the term “forms quickly” with reference to a skin-tightening film means that the film forms within less than about 20 minutes, such as less than about 15 minutes, or less than about 10 minutes, after the skin-tightening composition is applied to the skin.

As used herein, the term “blur” with regard to skin imperfections means that the visual appearance of the imperfection is less noticeable.

As used herein, the term “tighten” means that the film contracts in a manner that skin has a tighter feel to the user, and that reduces the visual appearance of wrinkles in the skin.

As used herein, the term “soft focus” means that the visual appearance of the skin is more homogenous and matte, leading to the blurring or hiding of skin imperfections.

As used herein, “durable” means the film will not easily rub off, or will not be removed by sweat, water, makeup, lotions, or the like, such that the film will remain substantially intact until removed by the user.

### Systems

The systems according to embodiments of the disclosure comprise a cosmetic composition for making up the skin and a skin-tightening composition capable of forming a film on the skin.

### Cosmetic Compositions for Making-Up the Skin

According to various embodiments, the cosmetic compositions for making up the skin useful in the systems and methods described herein may be chosen from make-up, foundation, and concealer formulations. In various embodiments, the formulations may be chosen from liquid, cream, gel, mousse, stick, solid, and powder formulations.

The cosmetic compositions for making up the skin comprise one or more colorants, and optionally one or more volatile solvents. In at least some embodiments, the cosmetic compositions are free or substantially free of solvents altogether, such as, for example, when powder formulations are chosen.

Colorants

The cosmetic compositions for making up the skin comprise at least one colorant component. In various embodiments, the colorant component comprises organic pigments. In further embodiments, the colorant component consists essentially of organic pigments. In yet further embodiments, the colorant component consists of organic pigments.

The organic pigments may be present in the cosmetic composition in an amount ranging from about 2% to about 90%, such as about 5% to about 80%, by weight, relative to the cosmetic composition. In embodiments where there is no, or substantially no, solvent in the cosmetic composition, the amount of organic pigment may be higher. For example, the total amount of organic pigments may be about 50% to about 90% by weight, relative to the cosmetic composition. In embodiments where the cosmetic composition comprises one or more solvents, the amount of organic pigment may be lower. For example, the total amount of organic pigments may be about 10% to about 20% by weight, relative to the cosmetic composition.

By way of non-limiting example only, organic pigments that may be used include nitroso, nitro, azo, xanthene, pyrene, quinoline, anthraquinone, triphenylmethane, fluorane, phthalocyanin, metal complex, isoindolinone, isoindoline, quinacridone, perinone, perylene, diketopyrrolopyrrole, indigo, thioindigo, dioxazine, triphenylmethane and quinophthalone compounds. For example, the organic pigments may be chosen from carmine lake, carbon black, aniline black, azo yellow, quinacridone, phthalocyanine blue, the blue pigments codified in the Color Index under the references CI 42090, 69800, 69825, 73000, 74100 and 74160, the yellow pigments codified in the Color Index under the references CI 11680, 11710, 15985, 19140, 20040, 21100, 21108, 47000 and 47005, the green pigments codified in the Color Index under the references CI 61565, 61570 and 74260, the orange pigments codified in the Color Index under the references CI 11725, 15510, 45370 and 71105, the red pigments codified in the Color Index under the references CI 12085, 12120, 12370, 12420, 12490, 14700, 15525, 15580, 15620, 15630, 15800, 15850, 15865, 15880, 17200, 26100, 45380, 45410, 58000, 73360, 73915 and 75470, and the pigments obtained by oxidative polymerization of indole or phenolic derivatives as described in patent FR 2 679 771.

In at least certain embodiments, the cosmetic composition for making up the skin does not comprise inorganic pigments.

Solvents

In various embodiments, the cosmetic compositions for making up the skin comprise at least one volatile solvent, and optionally at least one non-volatile solvent. The cosmetic compositions may, in at least certain embodiments, comprise volatile and non-volatile solvents in a ratio of greater than about 5:1 volatile solvents:non-volatile solvents, such as greater than about 6:1, greater than about 7:1, greater than about 8:1, greater than about 9:1, or greater than about 10:1.

When present, the total amount of solvents (volatile and non-volatile) may be present in the cosmetic composition in an amount ranging from about 10% to about 40%, such as from about 15% to about 30%, by weight, relative to the cosmetic composition. When present, the volatile solvent may be present in the cosmetic composition in an amount ranging from about 10% to about 30%, such as from about 15% to about 25%, by weight, relative to the cosmetic composition.

The volatile solvents useful in the cosmetic compositions for making up the skin may, for example, be chosen from water, volatile hydrocarbon-based oils, volatile silicone oils, for example optionally comprising alkyl or alkoxy groups that are pendant or at the end of a silicone chain, volatile fluoro oils, and mixtures thereof. Apolar volatile solvents can also be used.

Exemplary volatile hydrocarbon oils include, but are not limited to, those having from 8 to 16 carbon atoms and their mixtures, such as branched C₈ to C₁₆ alkanes and C₈ to C₁₆ isoalkanes (also known as isoparaffins), isododecane, isodecane, isohexadecane. For example, the at least one solvent may be chosen from the oils sold under the trade names of Isobar® or Permethyl®, the C₈ to C₁₆ branched esters such as isohexyl or isodecyl neopentanoate and their mixtures. In at least certain embodiments, the volatile hydrocarbon oils have a flash point of at least 40° C. It is also possible to use mixtures of isoparaffins and other volatile hydrocarbon-based oils, such as petroleum distillates.

Further, volatile silicone oils may be chosen from linear or cyclic silicone oils, such as those having a viscosity at room temperature (25° C.) of less than or equal to 6 cSt and having from 2 to 7 silicon atoms, these silicones being optionally substituted with alkyl or alkoxy groups of 1 to 10 carbon atoms. Examples of volatile silicone oils that may be used include, but are not limited to, octamethyltetrasiloxane, decamethylcyclo-pentasiloxane, dodecamethylcyclohexasiloxane, heptamethyloctyltrisiloxane, hexamethyldisiloxane, decamethyltetrasiloxane, dodecamethylpentasiloxane, and their mixtures. In at least certain embodiments, the volatile silicone oils have a flash point of at least 40° C.

Non-volatile solvents useful according to the disclosure include but are not limited to polar oils such as hydrocarbon-based oils of animal origin, for example perhydrosqualene; hydrocarbon-based plant oils such as liquid triglycerides of fatty acids and of glycerol, in which the fatty acids may have varied chain lengths, these chains being linear or branched, and saturated or unsaturated, which can be chosen, for example, from wheatgerm oil, sunflower oil, corn oil, soybean oil, marrow oil, grapeseed oil, blackcurrant seed oil, sesame oil, hazelnut oil, apricot oil, macadamia oil, castor oil, avocado oil, karite butter, sweet almond oil, cotton oil, alfalfa oil, poppy oil, pumpkin oil, evening primrose oil, millet oil, barley oil, quinoa oil, olive oil, rye oil, safflower oil, candlenut oil, passion flower oil, musk rose oil and caprylic/capric acid triglycerides such as those sold by the company Stearineries Dubois or those sold under the names Miglyol 810, 812 and 818 by the company Dynamit Nobel; natural or synthetic esters of formula R¹COOR², wherein R¹ is a higher fatty acid residue comprising 7 to 19 carbon atoms, and R² is a branched hydrocarbon-based chain comprising 3 to 20 carbon atoms, such as, for example, purcellin oil (cetostearyl octanoate), isopropyl myristate and alkyl or polyalkyl octanoates, decanoates or ricinoleates; synthetic ethers of formula R³COR⁴, wherein R³ is a C₃ to C₁₉ alkyl radical, and R⁴ is a C₃ to C₂₀ alkyl radical; fatty alcohols comprising at least 12 carbon atoms, such as octyldodecanol or oleyl alcohol; cyclic hydrocarbons such as (alkyl)cycloalkanes, wherein the alkyl chain is linear or branched, saturated or unsaturated and comprises 1 to 30 carbon atoms, such as cyclohexane or dioctyl-cyclohexane; aromatic hydrocarbons, for example, alkenes such as benzene, toluene, 2,4-dimethyl-3-cyclohexene, dipentene, p-cymene, naphthalene or anthracene, and esters such as isostearyl benzoate; primary, secondary or tertiary amines such as triethanolamine; and mixtures thereof.

Film Formers

In various embodiments, the cosmetic compositions for making up the skin further comprise at least one film former. The term “film former” and variations thereof, as used herein, means a polymer or resin that leaves a film on the substrate to which it is applied, for example, after a solvent accompanying the film former has evaporated, absorbed into and/or dissipated on the substrate.

Without intending to be limiting, film forming polymers and resins may be chosen from any film forming polymer or resin capable, by itself or in the presence of an auxiliary film-forming agent, of forming a film that adheres to a support and especially to the skin. Among the film-forming polymers that may be used, mention may be made of synthetic polymers, of free-radical type or of polycondensate type, polymers of natural origin and mixtures thereof, such as acrylic polymers, polyurethanes, polyesters, polyamides, polyureas, cellulose-based polymers, for instance nitrocellulose, and latex film forming polymers. For example, latex film forming polymers may be chosen from acrylate latex polymers, polyurethane latex polymers, and mixtures thereof. As still further examples of film forming polymers, latex film forming polymers may be chosen from at least one random styrene acrylate copolymer or derivative thereof, and at least one acrylate copolymer or derivative thereof, and mixtures thereof. Further, resinous plant extracts such as rosin and shellac, or derivatives thereof, epoxy ester resins, polyvinylpyrrolidone (PVP), polyvinylpyrrolidone/vinyl acetate copolymers and vinyl acetate/crotonic acid copolymers, and silicone resins may also be mentioned

By way of examples, suitable film forming polymers and resins include sulfopolyester resins, such as AQ sulfopolyester resins, for example AQ29D, AQ35S, AQ38D, AQ38S, AQ48S, and AQ55S (available from Eastman Chemicals), Vinex resins, such as Vinex 2034, Vinex 2144, and Vinex 2019 (available from Air Products), Dermacryl acrylic resins (available from National Starch), polyvinlypyrrolidinones (PVP) such as Luviskol K17, K30, and K90 (available from BASF), water soluble copolymers of PVP, including PVP/VA S-630 and W-735 and PVP/dimethylaminoethylmethacrylate, and Copolymers such as Copolymer 845 and Copolymer 937 available from ISP.

The film former may be present in the cosmetic composition in an amount ranging from about 1% to about 20%, such as from about 5% to about 15%, by weight, relative to the cosmetic composition.

The cosmetic composition may further comprise any component traditionally used in compositions for making up the skin, including but not limited to surfactants, co-solvents (volatile and/or non-volatile), waxes, plasticizers, preservatives, fillers, active ingredients used to treat skin, and sunscreens. Of course, the skilled artisan will take care to choose components that do not, or do not substantially, negatively affect the skin-tightening properties of the film produced by the skin-tightening compositions of the systems and methods described herein.

In various embodiments, the cosmetic composition for making up the skin may be chosen from any known commercial make-up, foundation, or concealer formulation comprising one or more organic pigments and optionally one or more volatile solvents.

### Skin-Tightening Compositions

According to various embodiments, the skin-tightening compositions useful in the systems and methods described herein comprise at least one thermoplastic elastomer, at least one adhesive polymer, and at least one filler, which together form an association to form a skin-tightening film on the skin upon application. Additional optional components, such as solvents, silicone elastomers, humectants, and water, may also be included in the skin-tightening compositions according to embodiments of the disclosure.

Thermoplastic Elastomer

According to various embodiments, the at least one thermoplastic elastomer is chosen from amorphous hydrocarbon block copolymers of styrene and monomers of hydrocarbon containing 2 to 5 carbon atoms and comprising one or two ethylenic unsaturations, and having at least two glass transition temperatures (“T_{g}”). The block copolymers may be hydrocarbon-soluble or dispersible in the oily phase.

Non-limiting examples of unsaturated hydrocarbon monomers having 2 to 5 unsaturated carbon atoms include ethylene, propylene, butadiene, isoprene or pentadiene. In various exemplary and non-limiting embodiments, block copolymers may be chosen from those comprising at least one styrene block and at least one block comprising units selected from butadiene, ethylene, propylene, butylene, isoprene, or mixtures thereof. The hydrocarbon-based block copolymer may, for example, be an optionally hydrogenated copolymer comprising styrene blocks and ethylene blocks/C₃-C₄ alkylene or isoprene blocks.

The amorphous hydrocarbon block copolymers comprise at least one first block whose T_{g} is below about 20° C., such as below about 0° C., below about −20° C., or below about −40° C. The T_{g} of the first block can, for example, range from about −150° C. to about 20° C., such as from about −100° C. to about 0° C. The block copolymers also comprise at least one second block whose T_{g} is greater than about 25° C., such as greater than about 50° C., greater than about 75° C., greater than about 100° C., or greater than about 150° C. The T_{g} of the second block can, for example, range from about 25° C. to about 150° C., such as from about 50° C. to about 125° C., about 60° C. to about 120° C., or about 70° C. to about 100° C.

Exemplary, non-limiting amorphous diblock copolymers may be chosen from styrene-ethylene/propylene copolymers, styrene-ethylene/butadiene copolymers, styrene-ethylene/butylene copolymers, styrene-butadiene, or styrene-isoprene copolymers. Diblock copolymers are sold, for example, under the name Kraton® G1701E by Kraton Polymers.

Exemplary, non-limiting amorphous triblock amorphous copolymers may be chosen from styrene-ethylene/propylene-styrene copolymers, styrene-ethylene/butadiene-styrene copolymers, copolymers of styrene-isoprene-styrene, and copolymers of styrene-butadiene-styrene, such as those sold under the names Kraton® G1650, Kraton® D1101, D1102 Kraton®, Kraton® D1160 by Kraton Polymers. In one exemplary embodiment, the thermoplastic elastomer may be a mixture of a triblock copolymer styrene-butylene/ethylene-styrene diblock copolymer and a styrene-ethylene/butylene, such as those sold under the name Kraton® G1657M by Kraton Polymers.

The thermoplastic elastomer may be present in the skin-tightening composition in an amount up to about 25%, such as an amount ranging from about 5% to about 20%, about 6% to about 18%, about 7% to about 16%, about 8% to about 15%, about 9% to about 14%, by weight, relative to the weight of the composition.

Adhesive Polymer

Skin-tightening compositions according to the disclosure further comprise at least one adhesive film-forming polymer chosen from polymer particles of C₁-C₄ alkyl(methacrylate)polymer, stablilized in a non-aqueous dispersion, referred to herein for ease of reference as an “oil dispersion,” such as those described in WO2015/091513 which is incorporated by reference herein.

By way of example, the C₁-C₄ alkyl(meth)acrylate monomers may be chosen from methyl(meth)acrylate, ethyl(meth)acrylate, n-propyl(meth)acrylate, isopropyl(meth)acrylate, n-butyl(meth)acrylate and tert-butyl(meth)acrylate. For example, the polymer may be a methyl acrylate and/or ethyl acrylate polymer.

The polymer may also comprise an ethylenically unsaturated acid monomer or the anhydride thereof, chosen especially from ethylenically unsaturated acid monomers comprising at least one carboxylic, phosphoric or sulfonic acid function, such as crotonic acid, itaconic acid, fumaric acid, maleic acid, maleic anhydride, styrenesulfonic acid, vinylbenzoic acid, vinylphosphoric acid, acrylic acid, methacrylic acid, acrylamidopropanesulfonic acid or acrylamidoglycolic acid, and salts thereof. For example, the ethylenically unsaturated acid monomer may be chosen from (meth)acrylic acid, maleic acid, and maleic anhydride.

The salts may be chosen from salts of alkali metals, for example sodium or potassium; salts of alkaline-earth metals, for example calcium, magnesium or strontium; metal salts, for example zinc, aluminum, manganese or copper; ammonium salts of formula NH⁺, quaternary ammonium salts; salts of organic amines, for instance salts of methylamine, dimethylamine, trimethylamine, triethylamine, ethylamine, 2-hydroxyethylamine, bis(2-hydroxyethyl)amine or tris(2-hydroxyethyl)amine; lysine or arginine salts.

The polymer of the particles of the oil dispersion may thus comprise or consist essentially of about 80% to about 100%, by weight, of C₁-C₄ alkyl(meth)acrylate and of about 0% to about 20%, by weight, of ethylenically unsaturated acid monomer, relative to the total weight of the polymer. According to one exemplary embodiment, the polymer consists essentially of a polymer of one or more C₁-C₄ alkyl(meth)acrylate monomers. According to another exemplary embodiment, the polymer consists essentially of a copolymer of C₁-C₄ (meth)acrylate and of (meth)acrylic acid or maleic anhydride.

By way of non-limiting example only, the polymer of the particles in the oil dispersion, which may optionally be crosslinked or alternatively may not be crosslinked, may be chosen from methyl acrylate homopolymers, ethyl acrylate homopolymers, methyl acrylate/ethyl acrylate copolymers, methyl acrylate/ethyl acrylate/acrylic acid copolymers, methyl acrylate/ethyl acrylate/maleic anhydride copolymers, methyl acrylate/acrylic acid copolymers, ethyl acrylate/acrylic acid copolymers, methyl acrylate/maleic anhydride copolymers, and ethyl acrylate/maleic anhydride copolymers.

The polymer of the particles in the dispersion may have a number-average molecular weight ranging from about 2000 to about 10,000,000, for example ranging from about 150,000 to about 500,000. The polymer particles may be present in the oil dispersion in a content ranging from about 20% to about 60%, for example about 21% to about 58.5%, about 30% to about 50%, about 35% to about 45%, or about 36% to about 42%, by weight, relative to the total weight of the oil dispersion.

The stabilizer in the oil dispersion may be an isobornyl(meth)acrylate polymer chosen from isobornyl(meth)acrylate homopolymer and statistical copolymers of isobornyl(meth)acrylate and of C₁-C₄ alkyl(meth)acrylate present in an isobornyl(meth)acrylate/C₁-C₄ alkyl(meth)acrylate weight ratio of greater than about 4, for example greater than about 4.5, or greater than about 5. For example, the weight ratio may range from about 4.5 to about 19, such as from about 5 to about 19, or from about 5 to about 12.

By way of example only, the stabilizer may be chosen from isobornyl acrylate homopolymers, statistical copolymers of isobornyl acrylate/methyl acrylate, statistical copolymers of isobornyl acrylate/methyl acrylate/ethyl acrylate, and statistical copolymers of isobornyl methacrylate/methyl acrylate.

In various embodiments, the stabilizer may have a number-average molecular weight ranging from about 10,000 to about 400,000, such as from about 20,000 to about 200,000.

In various embodiments, the combination of the stabilizer+polymer of the particles present in the oil dispersion comprises from about 10% to about 50%, such as about 15% to about 30%, by weight of polymerized isobornyl(meth)acrylate, and from about 50% to about 90%, such as about 70% to about 85%, by weight of polymerized C₁-C₄ alkyl(meth)acrylate, relative to the total weight of the combination of the stabilizer+polymer of the particles.

The oily medium of the oil dispersion comprises a hydrocarbon-based oil. The hydrocarbon-based oil is an oil that is liquid at room temperature (25° C.). The term “hydrocarbon-based oil” means an oil formed essentially from, or even consisting of, carbon and hydrogen atoms, and optionally oxygen and nitrogen atoms, and not containing any silicon or fluorine atoms. It may contain alcohol, ester, ether, carboxylic acid, amine and/or amide groups.

Exemplary and non-limiting embodiments of the hydrocarbon-based oil medium of the oil dispersion include hydrocarbon-based oils containing up to about 40, such as from 8 to 16 or from 8 to 14, carbon atoms. Optionally, the hydrocarbon-based oil is apolar. For example, the hydrocarbon based oil may be chosen from isododecane.

The oil dispersion may be prepared, for example, as described in WO2015/091513.

In various embodiments, the adhesive polymer may have a T_{g} greater than about 25° C., such as greater than about 50° C., greater than about 75° C., or greater than about 100° C., according to various embodiments.

The at least one adhesive polymer may be present in the skin-tightening composition in an amount up to about 25%, such as an amount ranging from about 5% to about 20%, about 6% to about 18%, about 7% to about 16%, about 8% to about 15%, about 9% to about 14%, or relative to the weight of the composition.

Fillers

The skin-tightening compositions may optionally comprise at least one filler. The fillers may be mineral or organic in nature, and of any shape. In various embodiments, the fillers may have a particle size greater than about 100 nm, and/or a specific surface area greater than about 200 m²/g.

By way of non-limiting example, fillers may be chosen from talc, mica, silica, silica surface-treated with a hydrophobic agent, fumed silica, kaolin, polyamide (Nylon®) powders (e.g. Orgasol® from Atochem), polyurethane powders, poly-β-alanine powder and polyethylene powder, powders of tetrafluoroethylene polymers (Teflon®), lauroyllysine, starch, boron nitride, hollow polymer microspheres such as those of polyvinylidene chloride/acrylonitrile, for instance Expancel® (Nobel Industrie) or of acrylic acid copolymers (Polytrap® from the company Dow Corning) and silicone resin microbeads (Tospearls® from Toshiba, for example), elastomeric polyorganosiloxane particles, precipitated calcium carbonate, magnesium carbonate, magnesium hydrogen carbonate, hydroxyapatite, hollow silica microspheres (Silica Beads® from Maprecos), glass or ceramic microcapsules, and metal soaps derived from organic carboxylic acids containing from 8 to 22 carbon atoms and preferably from 12 to 18 carbon atoms, for example zinc stearate, magnesium stearate or lithium stearate, zinc laurate or magnesium myristate.

In at least certain embodiments, the at least one filler may be chosen from hydrophobic silica aerogel particles. Silica aerogels are porous materials obtained by replacing (by drying) the liquid component of a silica gel with air. Hydrophobic silica aerogel particles useful according to embodiments of the disclosure include silylated silica (INCI name: silica silylate) aerogel particles. The preparation of hydrophobic silica aerogel particles that have been surface-modified by silylation is described more fully in U.S. Pat. No. 7,470,725, incorporated by reference herein.

In various embodiments, aerogel particles of hydrophobic silica surface-modified with trimethylsilyl groups may be chosen. For example, the aerogel sold under the name VM-2260® by the company Dow Corning, the particles of which have an average size of about 1000 microns and a specific surface area per unit of mass ranging from 600 to 800 m²/g, or the aerogel sold under the name VM-2270®, also by the company Dow Corning, the particles of which have an average size ranging from 5 to 15 microns and a specific surface area per unit of mass ranging from 600 to 800 m²/g, may be chosen. In other embodiments, the aerogels sold by the company Cabot under the names Aerogel TLD 201®, Aerogel OGD 201®, and Aerogel TLD 203®, CAB-O-SIL TS-530, CAB-O-SIL TS-610, CAB-O-SIL TS-720, Enova Aerogel MT 1100®, and Enova Aerogel MT 1200®, may be chosen.

Optionally, mixtures of fillers may be present in the skin-tightening compositions according to the disclosure. For example, a mixture of different aerogel particles, or of an aerogel and a different type of filler, may be used.

The at least one filler may be present in a total amount ranging from about 0.1% to about 20% by weight, for example from about 0.2% to about 15%, from about 0.5% to about 10%, or from about 1% to about 6%, by weight, relative to the total weight of the skin-tightening composition. In at least certain exemplary embodiments, the filler is present in an amount less than about 5%, such as less than about 4%, by weight, relative to the total weight of the skin-tightening composition. In one embodiment, the filler is present in an amount up to about 3% by weight, relative to the total weight of the skin-tightening composition.

Additional Components

The skin-tightening compositions according to the disclosure may optionally further comprise additional components, such as solvents, silicone elastomers, humectants, water, and pigments.

Solvents

The skin-tightening compositions may comprise at least one solvent. Optionally, the skin-tightening compositions may comprise at least one solvent chosen from solvents having a vapor pressure at room temperature (25° C.) of greater than about 100 Pa, such as greater than about 500 Pa, or greater than about 1000 Pa. In various embodiments, the composition is free or substantially free of solvents having a vapor pressure at room temperature (25° C.) of less than about 25 Pa. In further embodiments, the skin-tightening composition may comprise at least one solvent having a vapor pressure at room temperature (25° C.) of greater than about 100 Pa, such as greater than 500 Pa, or greater than 1000 Pa, and at least one solvent having a vapor pressure at room temperature (25° C.) of less than about 100 Pa, such as less than about 50 Pa, or less than about 25 Pa.

In various embodiments, the skin-tightening compositions comprise at least one volatile organic solvent. The volatile organic solvent may be chosen from, for example, volatile hydrocarbon-based oils and volatile silicone oils.

For example, volatile hydrocarbon oils include, but are not limited to, those having from 8 to 16 carbon atoms and their mixtures, such as branched C₈ to C₁₆ alkanes and C₈ to C₁₆ isoalkanes (also known as isoparaffins), isododecane, isodecane, isohexadecane. For example, the at least one solvent may be chosen from the oils sold under the trade names of Isopar® or Permethyl®, the C₈ to C₁₆ branched esters such as isohexyl or isodecyl neopentanoate and their mixtures. In at least certain embodiments, the volatile hydrocarbon oils have a flash point of at least 40° C. It is also possible to use mixtures of isoparaffins and other volatile hydrocarbon-based oils, such as petroleum distillates.

Further, volatile silicone oils may be chosen from linear or cyclic silicone oils, such as those having a viscosity at room temperature (25° C.) of less than or equal to 6 cSt and having from 2 to 7 silicon atoms, these silicones being optionally substituted with alkyl or alkoxy groups of 1 to 10 carbon atoms. Examples of volatile silicone oils that may be used include, but are not limited to, octamethyltetrasiloxane, decamethylcyclo-pentasiloxane, dodecamethylcyclohexasiloxane, heptamethyloctyltrisiloxane, hexamethyldisiloxane, decamethyltetrasiloxane, dodecamethylpentasiloxane, and their mixtures. In at least certain embodiments, the volatile silicone oils have a flash point of at least 40° C.

Additionally, the at least one volatile solvent may be chosen from polar volatile solvents, including but are not limited to, alcohols, volatile esters and volatile ethers.

The at least one solvent may be present in the skin-tightening composition in an amount up to about 95%, such as up to about 90%, up to about 85%, up to about 80%, up to about 75%, up to about 70%, up to about 65%, up to about 60%, up to about 55%, or up to about 50%, by weight of the composition. For example, the at least one solvent may be present in the skin-tightening composition in an amount ranging from about 40% to about 95%, such as about 50% to about 90%, or about 60% to about 85%, or about 65% to about 80%, by weight of the composition.

Silicone Elastomer

The skin-tightening composition may further optionally comprise at least one silicone elastomer. Surprisingly, in certain embodiments, the at least one silicone elastomer may improve properties such as the thickness and water-resistance of the skin-tightening film, without significantly affecting the mechanical or optical properties of the film. In other embodiments, the addition of at least one silicone elastomer may decrease wettability by sebum, which will help prevent the film from losing tightening properties. It may, in at least certain embodiments, be advantageous to choose a silicone elastomer having greater than 1% active material (AM), such as greater than 2% AM.

The at least one silicone elastomer may, for example, be chosen from at least one silicone crosspolymer dispersed in at least one oil. The at least one silicone crosspolymer may, in certain embodiments, be chosen from dimethicone crosspolymers, such as dimethicone/vinyl dimethicone crosspolymers and dimethicone/phenyl vinyl dimethicone crosspolymers. In other embodiments, the silicone cross-polymer may be modified by one or more groups chosen from alkyl, polyether, polyglycerin groups. For instance, the alkyl modified silicone cross-polymers may be chosen from vinyl dimethicone/lauryl dimethicone cross-polymers, cetearyl dimethicone cross-polymers, and C₃₀-C₄₅ alkyl cetearyl dimethicone cross-polymers. Non-limiting examples of polyether modified silicone cross-polymers include dimethicone/PEG-10/15 cross-polymers. Exemplary alkyl and polyether modified silicone cross-polymers may be chosen, for example, from PEG-10/lauryl dimethicone cross-polymers and PEG-15/lauryl dimethicone cross-polymers. Exemplary polyglycerin modified silicone cross-polymers include dimethicone/polyglycerin-3 cross-polymers and lauryl dimethicone/polyglycerin-3 cross-polymers.

In at least certain embodiments, the silicone polymers do not comprise polyethylene glycol or polypropylene groups, or hydrophilic moieties. Optionally, the silicone elastomer may be chosen from the silicone organic blends isododecane (and) dimethicone crosspolymer (18% AM) sold under the name EL-8040 ID or dimethicone/bis-isobutyl PPG-20 crosspolymer (17% AM in isododecane) sold under the name EL-8050 ID, by Dow Corning; or isododecane (and) vinyldimethyl/trimethylsiloxysilicate stearyl dimethicone crosspolymer (20% AM in isododecane), sold under the name GEL BELSIL RG90 by Wacker.

The silicone crosspolymer may be dispersed in at least one oil. In certain embodiments, the oil may be chosen from silicone oils, such as cyclic and linear organopolysiloxanes. Cyclic organopolysiloxanes may include, for example, cyclotetrasiloxane; cyclopentasiloxane; and methylated cyclic organopolysiloxanes, for example, octamethylcyclotetrasiloxane and decamethylcyclopentasiloxane. Non-limiting examples of linear organopolysiloxanes include low molecular weight dimethicones; high molecular weight dimethicones; alkyl derivatives of linear organopolysiloxanes, for example, cetyl dimethicone and lauryl trimethicone; aryl derivatives of linear organopolysiloxanes, for example, phenyl trimethicone; and hydroxylated derivatives of linear organopolysiloxanes, for example, dimethiconol. In other embodiments, the oil may be chosen from organic oils, such as mineral oil; linear and branched alkanes, for example, isododecane; triethylhexanoin; and squalane.

The at least one silicone crosspolymer may, in some embodiments, comprise from about 5% to about 35% by weight, relative to the total weight of the silicone elastomer blend, for example, from about 10% to about 20% by weight, or from about 25% to about 35% by weight, or from about 20% to about 30% by weight. The at least one oil may comprise from about 65% to about 95% by weight, relative to the total weight of the silicone elastomer blend, such as from about 80% to about 90% by weight, or from about 65% to about 75% by weight, or from about 70% to about 80% by weight.

In various exemplary embodiments, the silicone elastomer blend comprises from about 20% to about 30% of dimethicone/vinyl dimethicone cross-polymer. In further exemplary embodiments, the silicone elastomer blend comprises from about 70% to about 80% by weight of dimethicone. In yet further exemplary embodiments, the silicone elastomer blend comprises from about 20% to about 30% of dimethicone/vinyl dimethicone cross-polymer and from about 70% to about 80% by weight dimethicone.

For example, silicone elastomers sold under the name KSG-16 dimethicone (and) dimethicone/vinyl dimethicone crosspolymer, KSG-21 (at 27% in active material) INCI name: Dimethicone/PEG-10 Dimethicone vinyl dimethicone crosspolymer), KSG-20 (at 95% % in active material) INCI name: PEG-10 Dimethicone Crosspolymer), KSG-30, (at 100% % in active material) INCI name: Lauryl PEG-15 Dimethicone vinyl dimethicone crosspolymer), KSG-31 (at 25% in active material) INCI name: Lauryl PEG-15 Dimethicone vinyl dimethicone crosspolymer), KSG-32 or KSG-42 or KSG-320 or KSG-30 (at 25% in active material) INCI name: Lauryl PEG-15 Dimethicone vinyl dimethicone crosspolymer), KSG-33: Lauryl PEG-15 (at 20% in active material) Dimethicone vinyl dimethicone crosspolymer), KSG-210 (at 25% in active material) INCI name: Dimethicone/PEG-10/15 crosspolymer), KSG-310: lauryl modified polydimethylsiloxane polyoxyethylenated in mineral oil, KSG-330 and KSG-340: PEG-15/lauryl dimethicone crosspolymer, and X-226146 (at 32% % in active material) INCI name: Dimethicone/PEG-10 Dimethicone vinyl dimethicone crosspolymer), all by Shin Etsu; DC9010 (at 9% in active material) and DC9011 (at 11% in active material) INCI name: PEG-12 dimethicone crosspolymer), DC9040 cyclopentasiloxane (and) dimethicone crosspolymer, and DC9041 dimethicone (and) dimethicone crosspolymer, all by Dow Corning; or the products sold under the VELVESIL product line by Momentive, such as VELVESIL 125 and VELVESIL DM, may be chosen.

Other examples of silicone elastomers include KSG-710 (at 25% in active material, INCI name: dimethicone/polyglycerin-3 crosspolymer); and KSG-820, KSG-830 and KSG-840, all of which are dimethicone/polvaleverin-3 crosspolymer (INCI), but in different diluents, 820 is in isododecane, 830 is in triethyl hexanoin, and 840 is in squalene, all by Shin Estu.

The at least one silicone elastomer may optionally be included in the skin-tightening composition in an amount up to about 10%, such as up to about 8%, up to about 5%, about 4.5%, up to about 4%, up to about 3.5%, up to about 3%, up to about 2.5%, up to about 2%, up to about 1.5%, up to about 1%, up to about 0.75%, up to about 0.5%, up to about 0.25%, up to about 0.2%, or up to about 0.1%, by weight, relative to the weight of the composition. In certain embodiments, the at least one silicone elastomer may be present in an amount ranging from about 1% to about 10%, such as about 2% to about 8%, about 3% to about 6%, or about 4% to about 5%, by weight, relative to the weight of the skin-tightening composition.

Humectants

Optionally, skin-tightening compositions according to the disclosure may comprise at least one humectant or moisturizing agent. Surprisingly, in at least certain embodiments, the at least one humectant may improve the optical properties and feeling of the film formed on the skin by the composition, without negatively affecting the skin-tightening properties of the film.

By way of example only, humectants or moisturizing agents may be chosen from polyhydroxy compounds including but not limited to glycerin and glycols such as, for example, propylene glycol, butylene glycol, dipropylene glycol and diethylene glycol, glycol ethers such as monopropylene, dipropylene and tripropylene glycol alkyl(C₁-C₄)ethers, monoethylene, diethylene and triethylene glycol.

The at least one humectant may be present in the skin-tightening composition in an amount up to about 20%, such as up to about 15%, up to about 14%, up to about 13%, up to about 12%, up to about 11%, up to about 10%, up to about 9%, up to about 8%, up to about 7%, up to about 6%, up to about 5%, up to about 4%, up to about 3%, up to about 2%, up to about 1%, or up to about 0.5%, by weight of the composition.

Water

Optionally, in at least certain embodiments, water may be added to the skin-tightening compositions according to the disclosure. Surprisingly, in certain non-limiting embodiments, water may improve the properties of the film formed on the skin by the composition, such as Young Modulus, transparency, cohesion, and thickness.

Water can be included in the skin-tightening composition in an amount up to about 15%, up to about 12%, up to about 10%, up to about 9%, up to about 8%, up to about 7%, up to about 6%, up to about 5%, up to about 4%, up to about 3%, up to about 2%, up to about 1%, or up to about 0.5%, by weight of the composition. In at least certain embodiments, the skin-tightening compositions are anhydrous or substantially anhydrous. In other embodiments, the skin-tightening compositions may be in the form of a water-in-oil (W/O) emulsion.

It may, in at least certain embodiments, be advantageous to include water and at least one humectant, for example water and glycerin, in the skin-tightening composition together.

### Skin-Tightening Film

When the skin-tightening compositions according to the disclosure are applied to the skin, the at least one thermoplastic elastomer, the at least one adhesive polymer, and the at least one filler together form a matrix that creates a film on the skin. The film formed by the compositions described herein form quickly, are long-lasting and durable, and have optical properties that are advantageous for a skin-tightening film, such as transparency, matte effect, and a soft focus effect which helps to blur skin imperfections so that they are less noticeable.

Additionally, as discussed above, the compositions according to the disclosure form a film that is stiffer than, and thus capable of tightening, human skin. Human skin has a Young Modulus in the range of 10 kPa to 100 kPa; thus, a film for tightening the skin should have a Young Modulus of greater than 100 kPa. The films that are formed by the compositions have Young Modulus' greater than 500 kPa (0.5 MPa) in some embodiments, greater than 1000 kPa (1 MPa) in some embodiments, greater than 5000 kPa (5 MPa) in some embodiments, and even greater than 10,000 kPa (10 MPa) in some embodiments. Additionally, the compositions according to the disclosure have sufficient consistency G* and phase angle below 45°, in order to form an effective and lasting film on the skin.

As such, the amounts and components of the composition should be chosen to provide a film on the skin that is capable of tightening the skin, while also blurring skin imperfections.

In various exemplary embodiments, for the best film properties, it may be advantageous for the total amount of thermoplastic elastomer plus adhesive polymer plus filler to be greater than about 10%, such as greater than about 15% or greater than about 20%, by weight, of the total weight of the composition.

In yet further exemplary embodiments, for the best film properties, it may be advantageous for amounts of the thermoplastic elastomer and adhesive polymer to be chosen so that the ratio of thermoplastic elastomer:adhesive polymer is in the range of about 1:10 to 10:1, in the range of about 1:5 to 5:1, or in the range of about 1:1 to 8:1.

The films may be formed quickly, for example within less than about 30 minutes, less than about 20 minutes, less than about 10 minutes, or less than about 5 minutes, after the composition is applied to the skin.

Films according to the disclosure may be long-lasting. For example, once the composition is applied to the skin and a film is formed, the film may remain substantially intact on the skin for a period of at least about 12 hours, such as at least about 24 hours, at least about 48 hours, or at least about 72 hours.

The films may also be durable. For example, the film may not rub off, may not come off with sweat, or when the film is contacted by water, makeup, lotions, or other products that the user may wish to put on the skin.

### Methods

Methods of improving the appearance of the skin using the systems described herein are also disclosed, said methods comprising applying a skin-tightening composition according to the disclosure onto the skin in order to form a film on the skin, either before or after applying a cosmetic composition to the skin to make up the skin. Methods comprise tightening the skin, e.g. to get rid of, or reduce the appearance of, wrinkles, eye bags, etc., while also blurring or hiding skin imperfections, e.g. to camouflage pimples, pores, dark spots, uneven pigmentation, etc.

Optionally, in various methods, the skin tightening composition of the systems described herein may be applied to the skin first as a base coat, followed by an optional drying time during which a skin-tightening film is formed on the skin, followed by applying the cosmetic composition onto the skin on top of the film.

In further methods, the cosmetic composition of the systems described herein may be applied to the skin first as a base coat, followed by an optional drying time to allow the cosmetic composition to dry, followed by applying the skin-tightening composition onto the skin on top of the cosmetic composition to form a skin-tightening film thereon.

Surprisingly and unexpectedly, the systems and methods described herein minimize negative impact of the cosmetic composition for making up the skin on the film that is formed on the skin. It has been found that the systems and methods described herein avoid or minimize the drawback of migration of pigments from the cosmetic composition to the film, and thus avoid or minimize the associated softening of the film which can diminish the film's skin-tightening properties.

Further, the systems and methods described herein provides for an appearance of the skin that has a substantially homogenous coloration that is long-lasting and durable. The systems and methods thus effectively hide skin imperfections while simultaneously minimizing the appearance of wrinkles. In at least certain embodiments, the appearance of skin imperfections, wrinkles, or both may be minimized to a greater extent with the systems and methods of the disclosure, as compared to either skin-tightening compositions or cosmetic compositions for making up the skin alone.

### Kits

The disclosure further relates to kits comprising, in one compartment, (1) a skin-tightening composition comprising (a) at least one amorphous hydrocarbon block copolymer of styrene and monomers of hydrocarbon containing 2 to 5 carbon atoms and comprising one or two ethylenic unsaturations, and having a first T_{g} below about 0° C., and a second T_{g} greater than about 25° C., (b) at least one adhesive film-forming polymer chosen from polymer particles of C₁-C₄ alkyl(methacrylate)polymer, stablilized in a non-aqueous dispersion, and (c) at least one filler; and in a second compartment (2) a cosmetic composition for making up the skin comprising at least one colorant and optionally at least one volatile solvent.

It to be understood that, as used herein the terms “the,” “a,” or “an,” mean “at least one,” and should not be limited to “only one” unless explicitly indicated to the contrary. Thus, for example, reference to “a portion” includes examples having two or more such portions unless the context clearly indicates otherwise.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that any particular order be inferred.

While various features, elements or steps of particular embodiments may be disclosed using the transitional phrase “comprising,” it is to be understood that alternative embodiments, including those that may be described using the transitional phrases “consisting” or “consisting essentially of,” are implied. Thus, for example, implied alternative embodiments to a method that comprises A+B+C include embodiments where a method consists of A+B+C and embodiments where a method consists essentially of A+B+C. As described, the phrase “at least one of A, B, and C” is intended to include “at least one A or at least one B or at least one C,” and is also intended to include “at least one A and at least one B and at least one C.”

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints.

It is understood that when an amount of a component is given, it is intended to signify the amount of the active material.

It should be understood that all patents and published patent applications referenced are incorporated herein in their entireties.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements. The example that follows serves to illustrate embodiments of the present disclosure without, however, being limiting in nature.

The compositions and methods according to the present disclosure can comprise, consist of, or consist essentially of the elements and limitations described herein, as well as any additional or optional ingredients, components, or limitations described herein or otherwise known in the art.

It will be apparent to those skilled in the art that various modifications and variations can be made in the delivery system, composition and methods of the invention without departing from the spirit or scope of the invention. Thus, it is intended that the present invention cover the modifications and variations of this invention provided that they come within the scope of the appended claims and their equivalents.

### EXAMPLES

The following Examples are provided for illustrative purposes only, and are not intended to be limiting.

In each of the following examples, the amounts of components given are in terms of active material (AM).

### Dynamic Mechanical Analysis (DMA)

The determination of Young Modulus of the films for all Examples was as follows. The film was made by using a draw down bar at 8″ to cast the solution on a Teflon plate and dried the film at 40° C. in an oven overnight. The DMA Q800FR from TA instruments was used to measure the stress-strain response of the dried film. The deformation was applied from 0% strain to 200% strain at a rate of 100% strain/min at 32° C. Then the Young Modulus of the film was determined from the slope of the stress-strain curve in the linear viscoelastic regime.

### Example 1

Systems and methods for improving the appearance of the skin were evaluated.

A skin-tightening composition according to the disclosure was prepared as follows. The thermoplastic elastomer, Kraton (25%), was dispersed in isoparaffin oil with a mechanical stirrer and heated to 90° C. Stirring continued at 90° C. for 1-2 hours until all Kraton polymer was dissolved and the polymer solution became clear. The desired amounts of adhesive polymer and silica silylate were added into the Kraton/isoparaffin oil solution at the specified ratios in a plastic container, and the solution was mixed with a high speed mixer at 2500 rpm/min for 5 minutes. The final solution was kept at room temperature and sealed to avoid the evaporation of solvents.

Table 1 shows the skin-tightening composition prepared according to the disclosure:

TABLE 1
Skin-tightening Composition

| Component | Amount |
|---|---|
| HYDROGENATED STYRENE/ | 11.1% |
| BUTADIENE COPOLYMER | |
| OIL DISPERSION | 11.1% |
| ISODODECANE (AND) DIMETHICONE | 4.5% |
| CROSSPOLYMER | |
| SILICA SILYLATE | 3.0% |
| C8-9 ISOPARAFFIN | 33.3% |
| ISODODECANE | 37.0% |
| Total | 100.0% |
| Young Modulus (37° C.) | 24 MPa |

The Young Modulus of the film formed from the skin-tightening composition in Table 1 is 24 MPa at 37° C., which is greater than the Young Modulus of skin. Thus, the skin-tightening composition is able to produce a film capable of tightening the skin to minimize the appearance of wrinkles, crow's feet, eye bags, etc.

The following Table 2 shows commercially available cosmetic compositions for making up the skin:

TABLE 2
Commercially Available Cosmetic Compositions for Making up the Skin

| | Cosmetic | Cosmetic | Cosmetic | Cosmetic |
|---|---|---|---|---|
| | Composition 1 | Composition 2 | Composition 3 | Composition 4 |
| Commercial Brand | Maybelline | Lancôme | Maybelline | L'Oréal Paris |
| Product Name | New Instant Age | Teint Idole Ultra | Fit Me | True Match |
| | Rewind | 24 hour makeup | | |
| Type | Concealer | Makeup | Foundation | Foundation |
| Formulation | Liquid | Liquid | Powder | Liquid |
| Ratio volatile:non- | All volatile | All volatile | No solvent | 24.5%/ |
| volatile solvent | 24.5% | 18.3% | | 4.5% = <5:1 |
| Pigments/fillers % | 15% | 12% | >80% | 5.9% |

The skin-tightening composition of Table 1 was evaluated in systems comprising the commercially available cosmetic compositions of Table 2. The systems and methods were evaluated both (i) with the skin-tightening composition applied as a base coat and allowed to dry, wherein a skin-tightening film was formed on the skin and the cosmetic composition applied on top of the film, and (ii) with the cosmetic composition applied to the skin as a base coat and the skin-tightening composition applied on top so that a skin-tightening film formed on top of the cosmetic composition.

The following Table 3 shows the evaluation of the systems comprising the skin-tightening composition of Table 1 with the different cosmetic compositions of Table 2:

TABLE 3
Evaluation of Systems Comprising Skin-Tightening Compositions
and Cosmetic Compositions for Making Up the Skin

| | | Ex. 1-1 | Ex. 1-2 | Ex. 1-3 | Ex. 1-C |
|---|---|---|---|---|---|
| Skin- | | Table 1 | Table 1 | Table 1 | Table 1 |
| Tightening | | Composition | Composition | Composition | Composition |
| Composition | | | | | |
| Cosmetic | | Cosmetic | Cosmetic | Cosmetic | Cosmetic |
| Composition | | Composition 1 | Composition 2 | Composition 3 | Composition 4 |
| Film as top | Aspect | Smooth, | Smooth, | Smooth, | Heterogeneous |
| coat with | | homogeneous | homogeneous | homogeneous | color |
| Cosmetic | | film | film | film | |
| Composition | Young | 17 MPa | 17 MPa | 20 MPa | 34 MPa |
| under | Modulus | | | | |
| | (37° C.) | | | | |
| Film as base | Aspect | Smooth, | Smooth, | Smooth, | Heterogeneous |
| coat with | | slightly less | slightly less | slightly less | color |
| Cosmetic | | homogeneous | homogeneous | homogeneous | |
| Composition | | color | color | color | |
| on top | Young | 3 MPa | 13 MPa | 12 MPa | 16 MPa |
| | Modulus | | | | |
| | (37° C.) | | | | |

The results in Table 3 demonstrate that the film formed by the skin-tightening composition of Table 1 maintains sufficient Young Modulus, even with the addition of the cosmetic compositions in the systems of Ex. 1-1, 1-2, 1-3, and 1-C, to tighten the skin. This result demonstrates that the systems according to the disclosure minimize the appearance of eye bags, eye wrinkles, and crow's feet.

The results also demonstrate that the systems comprising skin-tightening compositions and cosmetic compositions for making up the skin according to the disclosure provide smooth films having homogenous color, which are able to hide or blur skin imperfections such as pimples, age spots, uneven skin tone, etc.

Finally, as shown in Ex. 1-C, the results demonstrate that when the ratio of volatile solvent:non-volatile solvent dips below 5:1, the results start to diminish.

We claim:

1. A system for improving the appearance of skin, comprising:
(1) a skin-tightening composition comprising:
a. at least one thermoplastic elastomer chosen from amorphous hydrocarbon block copolymers of styrene and monomers of hydrocarbon containing 2 to 5 carbon atoms and comprising one or two ethylenic unsaturations, and having a first T_{g} below about 0° C., and a second T_{g} greater than about 25° C.;
b. at least one adhesive film-forming polymer chosen from polymer particles of C₁-C₄ alkyl(methacrylate)polymer, stablilized in a non-aqueous dispersion; and
c. at least one filler; and
(2) a cosmetic composition for making up the skin, comprising at least one organic pigment and optionally at least one volatile solvent.

2. The system of claim 1, wherein the at least one thermoplastic elastomer is present in the composition in an amount ranging from about 5% to about 25% by weight, relative to the total weight of the composition.

3. The system of claim 1, wherein the at least one adhesive polymer is chosen from polymer particles comprising about 80% to about 100%, by weight, of C₁-C₄ alkyl(meth)acrylate and of about 0% to about 20%, by weight, of ethylenically unsaturated acid monomer of C₁-C₄ alkyl(methacrylate) polymer in an oil dispersion.

4. The system of claim 1, wherein the polymer of the particles is chosen from:
polymers consisting of at one or more C₁-C₄ alkyl(methacrylate)polymer; and
polymers consisting essentially of a copolymer of C₁-C₄ (meth)acrylate and of (meth)acrylic acid or maleic anhydride.

5. The system of claim 1, wherein the C₁-C₄ alkyl(methacrylate)polymer is chosen from methyl(meth)acrylate, ethyl(meth)acrylate, n-propyl(meth)acrylate, isopropyl(meth)acrylate, n-butyl(meth)acrylate and tert-butyl(meth)acrylate polymers.

6. The system of claim 3, wherein the oil comprises hydrocarbon based oils comprising up to 40 carbon atoms.

7. The system of claim 1, wherein the at least one filler is present in the composition in an amount ranging from about 0.1% to about 20% by weight, relative to the weight of the composition.

8. The system of claim 1, wherein the at least one solvent is chosen from volatile organic solvents.

9. The system of claim 1, wherein the skin-tightening composition further comprises at least one additional component chosen from silicone elastomers, humectants, water, and colorants.

10. The system of claim 1, wherein the skin-tightening composition further comprises at least one silicone elastomer chosen from silicone crosspolymers dispersed in at least one oil.

11. The system of claim 10, wherein the silicone crosspolymers are chosen from dimethicone crosspolymers.

12. The system of claim 10, wherein the at least one silicone elastomer is present in the composition in an amount up to about 10% by weight, relative to the total weight of the composition.

13. The system of claim 1, wherein the cosmetic composition for making up the skin comprises organic pigments chosen from nitroso, nitro, azo, xanthene, pyrene, quinoline, anthraquinone, triphenylmethane, fluorane, phthalocyanin, metal complex, isoindolinone, isoindoline, quinacridone, perinone, perylene, diketopyrrolopyrrole, indigo, thioindigo, dioxazine, triphenylmethane and quinophthalone compounds.

14. The system of claim 1, wherein the cosmetic composition for making up the skin comprises:
at least one volatile solvent and at least one non-volatile solvent, and
at least one film-former,
wherein the ratio of volatile:non-volatile solvents is greater than about 5:1.

15. The system of claim 14, wherein the at least one volatile solvent is chosen from water, volatile hydrocarbon-based oils, volatile silicone oils, optionally comprising alkyl or alkoxy groups that are pendant or at the end of a silicone chain, volatile fluoro oils, and mixtures thereof.

16. The system of claim 15, wherein the cosmetic composition for making up the skin is chosen from makeup, foundation, and concealer formulations.

17. The system of claim 1, wherein the at least one thermoplastic elastomer, at least one adhesive polymer, and at least one filler are present in the skin-tightening composition in a combined amount of greater than about 10% by weight, relative to the weight of the composition.

18. The system of claim 1, wherein the ratio of thermoplastic elastomer:adhesive polymer in the skin-tightening composition is in the range of about 1:1 to 8:1.

19. The system of claim 1, wherein the skin-tightening composition has a consistency G* of greater than about 100 Pa (at 10% strain) and a phase angle below about 45°.

20. A method for improving the appearance of skin, said method comprising:
(1) forming a film on the skin by applying a skin-tightening composition to the skin, the skin-tightening composition comprising:
a. at least one thermoplastic elastomer chosen from amorphous hydrocarbon block copolymers of styrene and monomers of hydrocarbon containing 2 to 5 carbon atoms and comprising one or two ethylenic unsaturations, and having a first T_{g} below about 0° C., and a second T_{g} greater than about 25° C.;
b. at least one adhesive film-forming polymer chosen from polymer particles of C₁-C₄ alkyl(methacrylate)polymer, stablilized in a non-aqueous dispersion; and
c. at least one filler,
wherein the Young Modulus of the film formed on the skin is greater than about 500 kPa; and,
after an optional drying time:
(2) applying a cosmetic composition to the skin on top of the film, the cosmetic composition comprising at least one organic pigment and optionally at least one volatile solvent.

21. A method for improving the appearance of skin, said method comprising:
(1) applying a cosmetic composition to the skin, the cosmetic composition comprising at least one organic pigment and optionally at least one volatile solvent; and
after an optional drying time:
(2) forming a film on the skin by applying a skin-tightening composition to the skin, the skin-tightening composition comprising:
a. at least one thermoplastic elastomer chosen from amorphous hydrocarbon block copolymers of styrene and monomers of hydrocarbon containing 2 to 5 carbon atoms and comprising one or two ethylenic unsaturations, and having a first T_{g} below about 0° C., and a second T_{g} greater than about 25° C.;
b. at least one adhesive film-forming polymer chosen from polymer particles of C₁-C₄ alkyl(methacrylate)polymer, stablilized in a non-aqueous dispersion; and
c. at least one filler,
wherein the Young Modulus of the film formed on the skin is greater than about 500 kPa.

22. A kit for improving the appearance of the skin, comprising:
(1) in a first compartment, a skin-tightening composition comprising:
a. at least one thermoplastic elastomer chosen from amorphous hydrocarbon block copolymers of styrene and monomers of hydrocarbon containing 2 to 5 carbon atoms and comprising one or two ethylenic unsaturations, and having a first T_{g} below about 0° C., and a second T_{g} greater than about 25° C.;
b. at least one adhesive film-forming polymer chosen from polymer particles of C₁-C₄ alkyl(methacrylate)polymer, stablilized in a non-aqueous dispersion; and
c. at least one filler; and
(2) in a second compartment, a cosmetic composition for making up the skin, comprising at least one organic pigment and optionally at least one volatile solvent.
